(19) United States
(12) Patent Application Publication
(10) Pub. No.: US 2021/0170118 A1
(43) Pub. Date: Jun. 10, 2021

(54) Twist-Lock Inhaler

(71) Applicant: Optimist Inhaler, LLC, Flowery Branch, GA (US)

(72) Inventors: David N. Jackson, Flowery Branch, GA (US); Russel M. Smith, Flowery Branch, GA (US)

(21) Appl. No.: 17/115,881

(22) Filed: Dec. 9, 2020

Related U.S. Application Data
(60) Provisional application No. 62/945,420, filed on Dec. 9, 2019.

(51) Int. Cl. A61M 15/00

(52) U.S. Cl. CPC A61M 15/0001; A61M 2205/276; A61M 15/009; A61M 15/0025

(57) ABSTRACT

A pulmonary inhaler includes a dual-action “child-proof” lock that requires more than one manual action to unlock the inhaler to allow the user to perform the dispensing action. For example, the user may press a small button on the back of the lower housing while rotating the upper housing with respect to the lower housing. These separate user actions performed simultaneously to unlock the inhale are is difficult for a child to perform accidentally. This rotation also moves an integral door that covers the mouthpiece aperture. The integral mouthpiece door, which prevents debris from getting inside the inhaler when it is locked, avoids the need for separate cap or tethered mouthpiece cover. The dual-action locking feature requires a minimal number of parts and provides an easily visible indication that the actuator is in the “locked” state or the “unlocked” state.

### REFERENCE TO RELATED APPLICATION

This application claims priority to commonly owned U.S. Provisional Application Ser. No. 62/945,420 filed Dec. 9, 2019, which is incorporated by reference.

### TECHNICAL FIELD

The present invention is directed to metered dose pulmonary inhaler and, more particularly, to a twist-lock inhaler that requires more than one manual action to unlock the inhaler to allow the user to perform the dispensing action.

### BACKGROUND

Metered dose inhalers (MDIs), also referred to as pressurized metered dose inhalers (pMDIs), are used by patients and consumers to dispense liquid medications from a pressurized canisters into their lungs for inhalation for medical, recreational and other purposes. The most common use of an MDI is for dispensing an aerosolized medication in to the lungs of a patient suffering from an asthma attack. The MDI has been trusted by physicians and prescribed to patients for over 40 years. There are three main components to an MDI, (1) the molded plastic actuator that utilizes a small spray nozzle to aerosolize or atomize the medication that is to be inhaled, (2) the canister and valve assembly which contains and doses the medication when the dispensing valve is pressed, and (3) a cap that is installed on the actuator mouthpiece to prevent small objects and debris from collecting inside the actuator and potentially being inhaled by the user. MDIs are typically used to deliver medication until the canister contents are depleted. The actuator, the canister and valve assembly, and the cap are then discarded.

In many conventional pulmonary inhalers, the canister and valve assembly is installed in an “inverted” or “upside-down” position into the actuator leaving the bottom of the canister exposed from the top of the actuator. The bottom of the canister acts as the “button” that the user presses down (compress toward the actuator) to depress the valve into the canister to release a dose of the pressurized medication from the canister into the mouthpiece of the inhaler. Due to the bottom of the canister being exposed above the top of the actuator, the inhaler is vulnerable to accidentally activation causing inadvertent dispensing of the medication when the inhaler is stored in a user's pocket, purse or backpack. This wastes the medication in the canister and can result in an unexpectedly depleted canister just when the patient suffers an asthma attack and urgently needs the inhaler to work.

Conventional asthma rescue inhalers include complicated airflow control mechanisms that automatically open the inhaler in response to inhalation suction on the mouthpiece to avoid a mechanical unlocking procedure that could impose a dangerous delay when the inhaler is needed for an emergency asthma rescue. These airflow control mechanisms allow the user to inhale deeply while dispensing the medicant from the inhaler, which is the expected manner of use of an asthma rescue inhaler. European patent application number 97302728.7 (EP Pub. EP0808635); U.S. Pat. Nos. 5,263,475 and 6,823,86362; and U.S. Patent Pub. No. 2010/0242960 describe inhalers with complex locking and airflow control mechanisms. While these types of inhalers are suitable by asthma rescue, they present drawbacks when used to deliver other types of medication that do not require emergency rescue activation or deep inhalation during medication dispensing. Although non-rescue inhalers are not typically used in an emergency rescue situation, they may dispense medications that can be dangerous to children. A need therefore exists for inhalers for medical, recreational and other purposes with child-proof locks and airflow control mechanisms suitable for purposes other than asthma rescue.

### SUMMARY

The present invention meets the needs described above through a pulmonary inhaler that includes a dual-action “child-proof” lock that requires more than one manual action to unlock the inhaler to allow the user to manually perform the dispensing action. In a representative embodiment, the user unlocks the inhaler by pressing and holding a small button on the back of the lower housing while rotating the upper housing with respect to the lower housing. These separate manual actions performed simultaneously to unlock the inhaler are very difficult for a child to perform accidentally. Rotating the upper housing with respect to the lower housing also moves an integral door into a position that covers the mouthpiece aperture. The integral door, which prevents debris from getting inside the inhaler when it is locked, avoids the need for separate cap or tethered mouthpiece cover as is common for conventional inhalers. The dual-action locking feature requires a minimal number of parts and provides an easily visible indication that the actuator is either in the “locked” state or in the “unlocked” state.

It will be understood that specific embodiments may include a variety of features in different combinations, as desired by different users. The specific techniques and structures for implementing particular embodiments of the invention and accomplishing the associated advantages will become apparent from the following detailed description of the embodiments and the appended drawings and claims.

### BRIEF DESCRIPTION OF THE FIGURES

The numerous advantages of the twist-lock inhaler may be better understood with reference to the accompanying figures in which:

FIG. 1 is a front perspective view of a twist-lock inhaler in a locked state.

FIG. 2 is a side view of the twist-lock inhaler in the locked state.

FIG. 3 is a front view of the twist-lock inhaler in the locked state.

FIG. 4 is a front perspective view of the twist-lock inhaler in an unlocked state.

FIG. 5 is a side view of the twist-lock inhaler in the unlocked state.

FIG. 6 is a front view of the twist-lock inhaler in the unlocked state.

FIG. 7A is a front perspective view of the twist-lock inhaler in the unlocked state.

FIG. 7B is a front perspective view of the twist-lock inhaler in a partially unlocked state.

FIG. 7C is a front perspective view of the twist-lock inhaler in the fully unlocked state.

FIG. 8A is a rear perspective view of the twist-lock inhaler prior to compression in a dispensing action.

FIG. 8B is a rear perspective view of the twist-lock inhaler after compression in the dispensing action.

### DETAILED DESCRIPTION

This present invention may be embodied in a pulmonary metered dose inhaler (MDI) that includes a dual-action “child-proof” lock mechanism that requires more than one manual action to unlock the inhaler to allow the user to manually perform the dispensing action. Embodiments of the invention may be used in connection with the metered dose inhalers and related systems described in U.S. Pat. No. 10,449,310, which is incorporated by reference. The twist-lock inhaler is not intended for use by patients who are in an “emergency” or “rescue” situation, such as a patient suffering from an asthma attack. Emergency “rescue inhalers” utilize automatic unlock features activated by the user's inhalation to avoid the risk of delayed dispensing in an emergency situation. Embodiments of the present invention, on the other hand, are well suited to the delivery of other types of medicants, such as cannabis preparations, pain killers, nicotine for smoking cessation programs, appetite suppressants for weight loss, and other liquids for purposes other than asthma rescue.

In a representative embodiment, the user unlocks a “twist-lock” inhaler by pressing and holding a small button on the back of the lower housing while simultaneously rotating the upper housing with respect to the lower housing. The twist-lock inhaler includes an upper housing configured to be manually aligned with the lower housing to allow the housings to be compressed toward each other in the “dispensing action” used to release a metered dose of medication from the canister. When the upper and lower housings are manually aligned, eccentric ledges on the upper and lower housings are aligned to allow the housings to be compressed toward each other during the dispensing action. When the upper and lower housings are manually misaligned, the eccentric ledges are misaligned to physically interfere with each other to prevent the upper and lower housings from being compressed towards each other.

To unlock the twist-lock inhaler, the user presses and holds the release button while simultaneously rotating the upper housing with respect to the lower housing in a first rotational direction (e.g., counterclockwise) to place the inhaler in the unlocked state with the upper housing aligned with the lower housing. With the inhaler in the unlocked state, the upper and lower housings can be manually compressed toward each other to dispense the medication from the canister. The user can also manually twist the upper housing with respect to the lower housing in the opposite rotational direction (e.g., clockwise) to place the inhaler in the locked state with the upper housing misaligned with the lower housing. With the inhaler in the locked state, the eccentric ledges on the upper and lower housings interfere with each other to prevent the upper and lower housings from being manually compressed toward each other, which prevents the medication from being dispensed from the canister.

As a security feature, the twist-lock mechanism includes a first unlock feature that requires the user to release a latch, such as detent mechanism. While the user manually holds the latch in a released position, a second unlock features requires the user to manually twist the upper inhaler housing relative to the lower inhaler to align the housings. The dual-action twist-lock procedure provides a simple yet highly effective “child-proof” security feature requiring two separate, simultaneous physical actions to unlock the inhaler. In the representative embodiment, the release button is part of a detent mechanism that selectively latches the upper and lower housings to each other. The user presses the release button inward, and holds the release button in that position, while the user twists upper housing with respect to the lower housing to transition the inhaler out of the locked state to unlock the inhaler. A child is very unlikely to perform these combined actions accidentally. Similarly, the twist-lock inhaler is also very unlikely to be unlocked by accidental jostling while the inhaler is carried in a pocket, purse or backpack.

In the particular embodiment illustrated in the drawings, the release button is formed into the outer surface of the upper housing, while a button hole is formed into the lower housing. The button and button hole form a detent latch mechanism selectively securing the upper housing to the lower housing. The user manually depresses the button inward, and holds it in that released position, to release the button from the button hole to disengaged the detent mechanism. At the same time, the user rotates the upper housing of the actuator with respect to the lower housing to align the upper and lower housings to unlock the inhaler. Once the upper and lower housings are aligned, they can be compressed towards each other to depress a valve into the medication canister to dispense a metered dose of medication from the canister. The reverse rotation sequence reintroduces the interference mate between the upper and lower housing to return the inhaler to the locked state. In the locked state, the button protrudes from the upper housing and fits snugly into the button hole formed into the lower housing. The user manually presses and holds the button inward to release the detent mechanism allowing the user to manually rotate upper housing with respect to the lower housing. While the release button is a convenient type of detent latch mechanism, other types of latch mechanisms may be used, such as a tab, slider, clasp, pawl or other suitable type of latch.

To keep debris out of the inhaler, the twist action also rotates a mouthpiece door to cover the mouthpiece aperture when the inhaler is locked. In the representative embodiment, the mouthpiece door is a physical extension of the upper housing so that it rotates along with the upper housing. The door slides to uncover the mouthpiece aperture when the actuator is manually moved into the unlocked state for medication dispensing. As the upper housing is rotated in the opposite direction to lock the actuator, the door rotates in the opposite direction to seal off the mouthpiece aperture to prevent debris and other particulate from entering the mouthpiece when locked and not in use. Conventional actuators typically include a cap or cover for the mouthpiece configured as a separate part that is not attached to the mouthpiece. This creates a scenario where the cap is often lost or misplaced when removed for dispensing medication. The door that covers the mouthpiece aperture of the twist-lock inhaler is built into the inhaler, and thus not removable and subject to loss.

As the upper and lower housings are rotated in opposing directions, an audible “click” is produced so that the user can hear the lock feature engaging and disengaging. Thus, the upper and lower housing make a “click” sound and as the user manually rotates the housings to the “unlocked” state with the mouthpiece door open. Another audible “click” is produced when the user manually rotates the housings in the opposite direction to lock the inhaler with the door covering the mouthpiece aperture. The interfering upper and lower housings also provide an easily visible indication that the actuator is either in a “locked” or “unlocked” state. The user can easily identify the “locked” state by viewing the inhaler from the side to see if the upper housing is touching the lower housing. The opposite is observed in the “unlocked” state; allowing the user to view the inhaler from the side to easily see whether upper housing is not touching the lower housing. The user can also view the inhaler from the front to see whether the mouthpiece door is open or closed.

Conventional asthma rescue inhalers also include large air vents to allow for easy and unrestricted airflow through the inhaler when the user breathes in through the actuator mouthpiece. This is because the user is expected to inhale deeply while dispensing the medication from an asthma rescue inhaler. While this is a desirable feature for an asthma rescue inhaler, it presents disadvantages for other types of inhalers. More specifically, it has been discovered that reducing the air-flow velocity through inhalers (other than asthma rescue inhalers) helps to reduce throat irritation and coughing caused by the impaction of the dispensed medication on the back of the throat and in the upper respiratory tract. The twist-lock inhaler therefore eliminates or reduces the large air vents to reduce the airflow to decrease the user's inhalation rate and thereby reduce coughing verses an actuator with minimal airflow resistance. Reducing the airflow during dispensing may also improve the performance, efficiency and ability of the twist-lock inhaler to deliver more medication to the lungs versus a traditional asthma recue actuator. In a particular embodiment, the twist-lock inhaler may be designed to reduce the airflow through the actuator to limit the air exiting the mouthpiece to approximately 1 to 15 liters per minute.

The twist-lock design creates a low-airflow seal between the upper and lower canisters preventing over-inhalation during the dispensing action. As an option, the twist-lock inhaler may not include any additional air inlet vents to minimize the airflow created by user inhalation. In this case, the only areas in which the twist-lock inhaler allows air to flow into the inhaler are limited to the low-airflow seal, part lines and gaps required for assembling the parts of the device. The twist-lock inhaler may therefore be designed to virtually omit all inlets, vents, and large holes that allow air to easily enter into the inhaler to limit the inhaler to a very low airflow. As another option, one or more selected-size air vents may be included to provide a selected amount of airflow through the inhaler when it is in the unlocked position. To prevent contamination, the air vents may be covered when the inhaler is in the closed position, and uncovered when the inhaler is in the open position. In a particular embodiment, the air vents are selected to limit the airflow through the inhaler to 1 to 15 liters per minute.

The low-airflow seal and selected selected-size air vents also produce a relatively constant airflow velocity that does not vary significantly with changing suction caused by user inhalation.

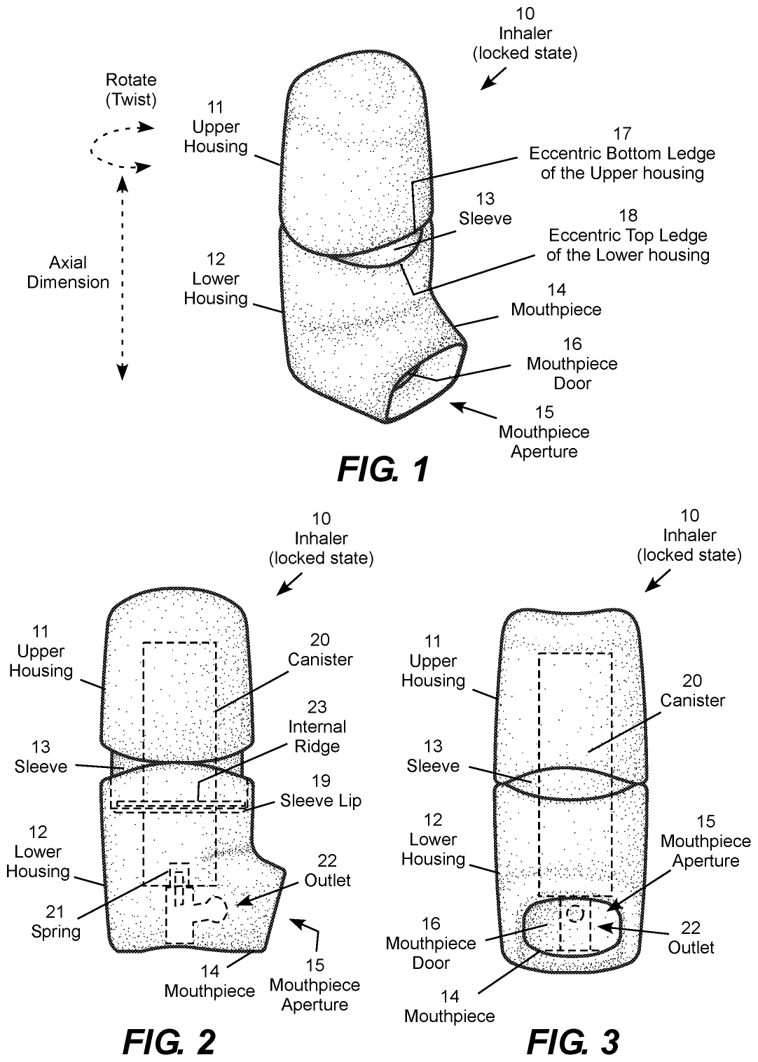

FIG. 1 is a front perspective view, FIG. 2 is a side view, and FIG. 3 is a front view of a twist-lock inhaler 10 in a locked state. The inhaler 10 includes an upper housing 11 and a lower housing 12. A sleeve 13 that extends from the upper housing 12 holds the upper and lower housings together while allowing them to rotate about an axial dimension (vertical in FIG. 1-3). The inhaler 10 includes a mouthpiece 14 with a mouthpiece aperture 15 that leads into the interior of the inhaler. A mouthpiece door 16, which is barely visible in FIG. 1, rotates with the upper housing to removably cover the mouthpiece aperture 15. A canister 20 inside the inhaler contains a pressurized medication or other substance to be dispensed (sprayed) through the mouthpiece aperture 15. A spring 21 forming part of the valve mechanism of the canister 20 biases the canister 20 away from the lower housing 12, which biases the upper housing 11 away from the lower housing along the axial dimension. When the user compresses the upper housing 11 toward the lower housing 12 along the axial dimension, a valve is depressed into the canister, which dispenses a metered dose of the contents of the canister 20 through an outlet 22 that is aligned with the mouthpiece aperture 15. The sleeve 13 also allows the upper and lower housings to reciprocate toward and away from each other along the axial dimension a sufficient amount to dispense metered doses of contents of the canister 20. Additional details of the inhaler and related systems are described in U.S. Pat. No. 10,449,310, which is incorporated by reference.

FIGS. 1-3 show the inhaler 10 in the locked state. When the upper housing 11 and the lower housing 12 are misaligned in the locked state, an eccentric bottom ledge 17 of the upper housing 11 interferes with an eccentric top ledge 18 of the lower housing 12 to prevent the upper and lower housings from being compressed towards each other, which prevents the contents of the canister 20 from being dispensed through the outlet 22. In addition, the mouthpiece door 16 covers the mouthpiece aperture 15 when the inhaler 10 is in the locked state to keep debris from entering the inhaler. In this embodiment, a sleeve lip 19 extending radially outward from the sleeve 13 engages with an internal ridge 23 on the inside wall of the lower housing 12 to capture the upper housing 11 while allowing the upper and lower housings to rotate and reciprocate with respect to each other. The spring 21 that biases the canister 20 and the upper housing 11 away from the lower housing 12 also biases the sleeve lip 19 against the internal ridge 23 to maintain the lip in engagement with the internal ridge.

Also in this embodiment, the sleeve 13 and the door 16 are physical extensions of the upper housing 12 and therefore rotate with the upper housing. This causes the door 16 to be manually moved to cover the mouthpiece aperture 15 as the upper housing is manually rotated with respect to the lower housing to place the inhaler in the locked state. Similarly, the door 16 is manually moved to uncover the mouthpiece aperture 15 as the upper housing is manually rotated with respect to the lower housing to place the inhaler in the unlocked state.

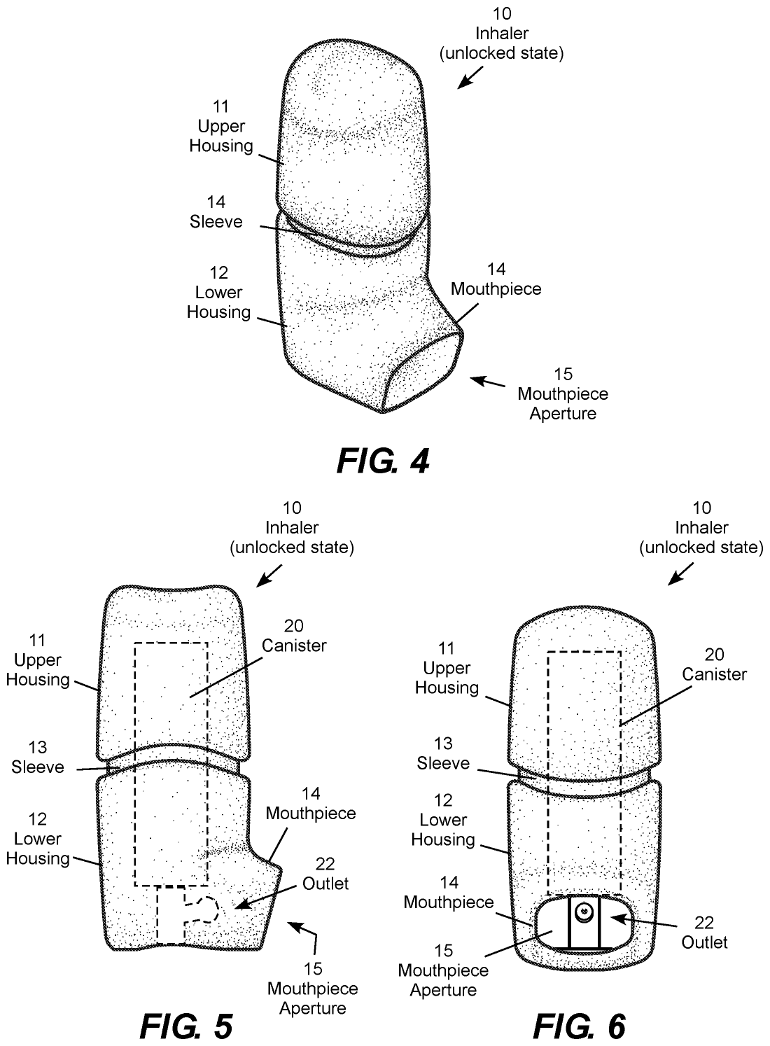

The user rotates (twists) the upper housing 11 with respect to the lower housing 12 about the axial dimension to unlock the inhaler. FIGS. 4-6 show the inhaler 10 in the unlocked state, in which the upper and lower housings 11, 12 are aligned to allow the upper and lower housings to be compressed towards each other along the axial dimension to dispense the contents of the canister 20 from the outlet 22. The mouthpiece door 16 rotates with the upper housing 12 to uncover the mouthpiece aperture 15 when the inhaler 10 is in the unlocked state. The user may then reciprocate (pump) the inhaler as desired to deliver the desired number of metered doses of the contents of the canister 20 out of the outlet 22, through the mouthpiece aperture 15, and into the user's lungs.

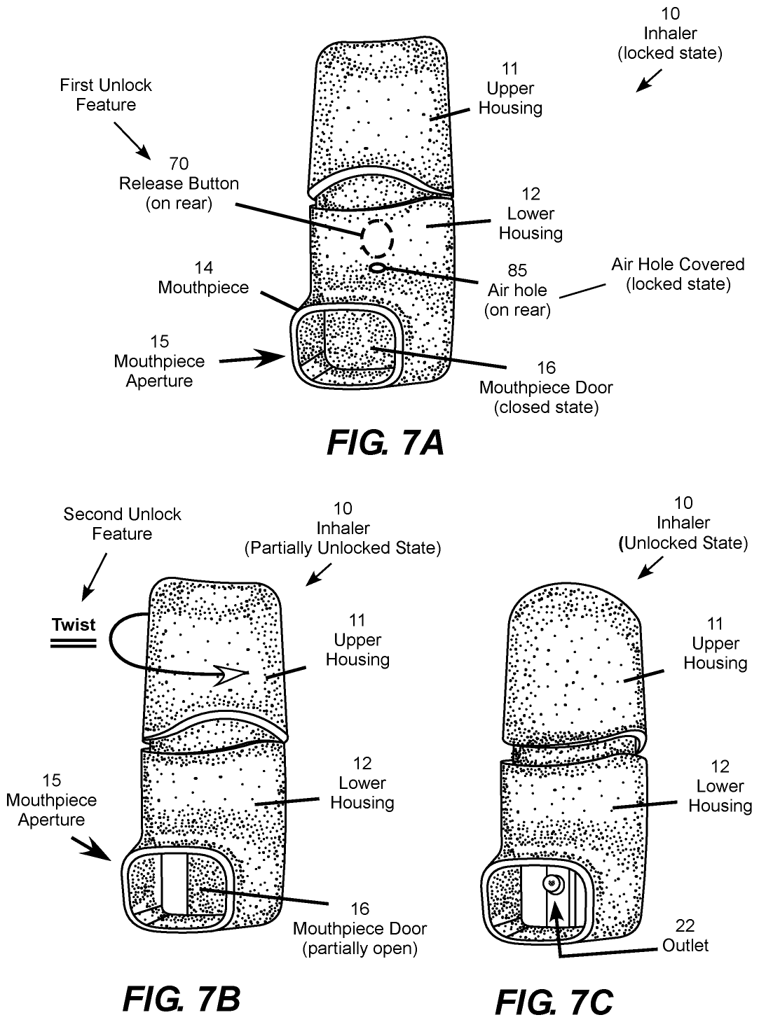

FIGS. 7A, 7B and 7C are a sequential series of drawings illustrating the transition of the inhaler 10 from the locked state (FIG. 7A) through a partially unlocked state (FIG. 7B) to the fully unlocked state (FIG. 7C). FIGS. 7A-7C also illustrate the dual-action security feature producing a very effective child-proof lock. As shown in FIG. 7A, the inhaler 10 includes a detent mechanism, in this embodiment a release button 70, which releasably latches the upper housing 11 to the lower housing 12. As a first manual action required to unlock the inhaler, the user depresses and holds the release button 70 to unlatch the detent mechanism. This allows the user to simultaneously perform a second unlocking action, in this example rotating (twisting) the upper housing 11 with respect to the lower housing 12.

FIG. 7A shows the upper housing 11 fully misaligned with the lower housing 12 (locked state), FIG. 7B shows the upper housing partially rotated with respect to the lower housing (partially unlocked state), and FIG. 7C shows the upper housing fully aligned with respect to the lower housing (unlocked state). The rotation of the upper housing 11 with respect to the lower housing 12 to unlock the inhaler also rotates the door 16 to uncover the mouthpiece aperture 15. FIG. 17A shows the door 16 fully covering the mouthpiece aperture 15 (locked state), FIG. 7B shows the door 16 partially covering the mouthpiece aperture (partially unlocked state), and FIG. 7C shows the door 16 fully uncovering the mouthpiece aperture (unlocked state). In the unlocked state shown in FIG. 7C, the outlet 22 is directed toward the mouthpiece aperture 15 for dispensing (spraying) the contents of the canister 20 through the mouthpiece aperture and into the lungs of the user. The dual-action twist-lock requires the user to perform two manual operations, namely pressing the release button and twisting the housings, at the same time to unlock the inhaler. This dual-action twist lock produces a very simple yet effective child-proof lock. The dual-action twist-lock is also very unlikely to be inadvertently unlocked by jostling while carrying the inhaler in a pocket, purse or backpack.

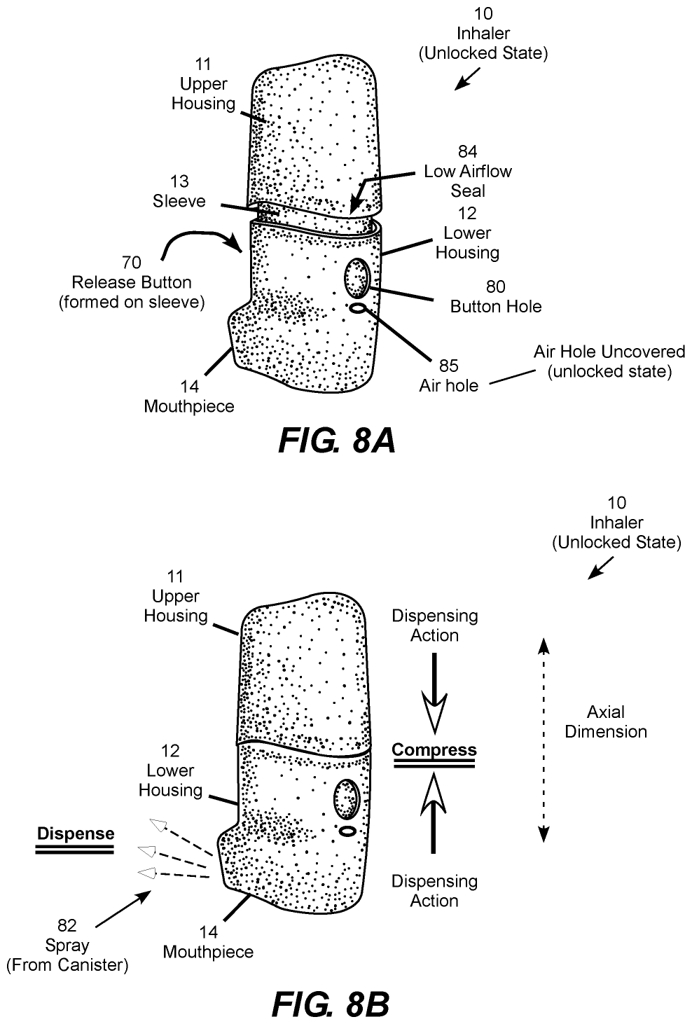

FIG. 8A is a rear perspective view of the twist-lock inhaler 10 in the unlocked state prior to the dispensing action. As described above, the user depresses and holds the release button 70 to unlatch the detent mechanism. In this particular embodiment, the release button 70 is a protrusion formed into the sleeve 13 of the upper housing 11. In addition, the lower housing 12 includes a button hole 80 sized to releasably receive the button 70 to form a detent mechanism. The sleeve 13 is sufficiently pliable to allow the user to press the release button 70 sufficiently into the button hole 80 of the lower housing 12 to unlatch the upper housing 11, which allows the user to then rotate the upper housing with respect to the lower housing. As shown in FIG. 8B, once the inhaler is in the unlocked state, the user can compress the upper housing 11 toward lower housing 12 along the axial dimension to perform the dispensing action required to release a desired metered dose of spray 82 from the pressurized canister 20 through the mouthpiece 14 and into the lungs of the user.

Referring again to FIG. 7A, the inhaler is in the locked state when the eccentric bottom ledge 17 of the upper housing 11 is misaligned with the eccentric top ledge 18 of the lower housing 12. Similarly, the inhaler is in the unlocked state when the eccentric bottom ledge 17 of the upper housing 11 is aligned with the eccentric top ledge 18 of the lower housing 12 as shown in FIG. 8A.

FIGS. 8A and 8B also depict an airhole 85, which is an optional feature of the twist-lock inhaler 10. Generally, the upper and lower housings 11, 12 form a low-airflow seal 84 that restricts the ability of the user to inhale deeply by creating suction on the inhaler mouthpiece. If desired, the inhaler 10 may include one or more air release vents, represented by the airhole 85 in this embodiment, to allow the user to inhale at a desired rate while dispensing the contents of the canister. Without the airhole 85, the airflow through the is limited to the low-airflow seal 84 and other part lines and incidental leakage. The airhole 85 permits a selected airflow to be conducted through the inhaler to accommodate a desired dosing airflow rate, such as 1 to 15 liters per minute. In the representative embodiment, the sleeve 13 includes an air hole that becomes aligned with the airhole 85 through the lower housing 12 when the inhaler 10 is in the unlocked state. The air hole through the sleeve also becomes blocked by the lower housing 12 when the inhaler 10 is in the locked state. The size and location of the airhole 85 shown in the figures are merely demonstrative, and multiple air vents may be included in any number, size and location desired to achieve the desired airflow through the inhaler.

The upper housing 11, the lower housing 12, the sleeve 13, and the mouthpiece door 16 may be manufactured from a suitable plastic, such as Acrylonitrile butadiene styrene (ABS) or polycarbonate. The canister 20 may be a conventional stainless steel or aluminum medication canister currently used with asthma rescue and other types of inhalers for medical, recreational and other purposes. The outlet 22 and the other internal components of the twist-lock inhaler 10 may be similar to those found in conventional asthma rescue and other types of inhalers.

While particular aspects of the present subject matter have been shown and described in detail, it will be apparent to those skilled in the art that, based upon the teachings of this disclosure, changes and modifications may be made without departing from the subject matter described in this disclosure and its broader aspects and, therefore, the appended claims are to encompass within their scope all such changes and modifications as are within the true spirit and scope of the subject matter described in this disclosure. Although particular embodiments of this disclosure have been illustrated, it is apparent that various modifications and embodiments of the disclosure may be made by those skilled in the art without departing from the scope and spirit of the disclosure.

It is believed that the present disclosure and many of its attendant advantages will be understood by the foregoing description, and it will be apparent that various changes may be made in the form, construction and arrangement of the components without departing from the disclosed subject matter or without sacrificing all of its material advantages. The form described is merely explanatory, and it is the intention of the following claims to encompass and include such changes. The disclosure is defined by the following claims, which should be construed to encompass one or more structures or function of one or more of the illustrative embodiments described above, equivalents and obvious variations. The foregoing relates only to the exemplary embodiments of the present invention, and that numerous changes may be made therein without departing from the spirit and scope of the invention as defined by the following claims.

The invention claimed is:

1. A pulmonary inhaler, comprising:
an upper housing;
a lower housing configured to reciprocate with respect to the upper housing comprising a mouthpiece defining a mouthpiece aperture;
a canister containing a pressurized substance located within the upper and lower housings and positioned to dispense the pressurized substance into the mouthpiece when the upper housing is manually compressed toward the lower housing;
wherein the upper housing is configured for movement with respect to the lower housing to selectively transition the inhaler between a locked state and an unlocked state;
further comprising a dual-action twist lock that requires two manual actions to be performed simultaneously to transition the inhaler from the locked state to the unlocked state for dispensing the pressurized substance from the canister into the mouthpiece.

2. The pulmonary inhaler of claim 1, wherein the upper housing is configured for rotational movement with respect to the lower housing in a first rotational direction to transition the inhaler from the unlocked state to the locked state, and wherein the upper housing is configured for rotational movement with respect to the lower housing in a second rotational direction to transition the inhaler locked state to the unlocked state.

3. The pulmonary inhaler of claim 1, further comprising a mouthpiece door configured to move to cover the mouthpiece aperture as the inhaler is manually transitioned to the locked state, further configured to move to uncover the mouthpiece aperture as the inhaler is manually transitioned to the unlocked state.

4. The pulmonary inhaler of claim 3, wherein the mouthpiece door comprises a physical extension of the upper housing.

5. The pulmonary inhaler of claim 1, wherein:
the dual-action twist lock comprises an eccentric bottom ledge of the upper housing and an eccentric top ledge of the lower housing;
the inhaler is configured to be manually transitioned into the unlocked state by manually rotating the eccentric bottom ledge of the upper housing into alignment with the eccentric top ledge of the lower housing; and
the inhaler is configured to be manually transitioned into the locked state by manually rotating the eccentric bottom ledge of the upper housing into misalignment with the eccentric top ledge of the lower housing.

6. The pulmonary inhaler of claim 5, wherein the dual-action twist lock further comprises a latch configured to be manually held in a released position while the upper housing is manually rotated with respect to the lower housing to manually transition the inhaler into the unlocked state.

7. The pulmonary inhaler of claim 6, wherein the latch comprises a button detent mechanism.

8. The pulmonary inhaler of claim 1, further comprising a sleeve connecting the upper housing to the lower housing allowing the upper housing to reciprocate with respect to the lower housing along an axial dimension.

9. The pulmonary inhaler of claim 8, wherein the sleeve further allows the upper housing to rotate with respect to the lower housing about the axial dimension.

10. The pulmonary inhaler of claim 9, wherein the sleeve comprises a portion of a low-airflow seal restricting airflow through the inhaler.

11. The pulmonary inhaler of claim 1, further comprising an air vent sized to allow a selected airflow through the inhaler.

12. The pulmonary inhaler of claim 1, further comprising a spring biasing the canister and the upper housing away from the lower housing.
